(19) United States
(12) Patent Application Publication
(10) Pub. No.: US 2013/0254914 A1
(43) Pub. Date: Sep. 26, 2013

(54) SPINACH HYBRID VARIETY CALLISTO

(71) Applicant: Nunhems B.V., Nunhem (NL)

(72) Inventor: Jan Ane Dijkstra, Beegden (NL)

(21) Appl. No.: 13/892,677

(22) Filed: May 13, 2013

(51) Int. Cl. A01H 5/12

(52) U.S. Cl. CPC A01H 5/12
USPC 800/260; 800/298; 435/410; 435/6.11; 47/58.1 FV; 426/615

(57) ABSTRACT

The invention provides seed and plants of the spinach variety designated CALLISTO. The invention thus relates to the plants, seeds and tissue cultures of spinach variety CALLISTO, and to methods for producing a spinach plant produced by crossing a plant of spinach variety CALLISTO with itself or with another spinach plant, such as a plant of another variety. The invention further relates to seeds and plants produced by such crossing. The invention further relates to parts of a plant of spinach variety CALLISTO, including the leaves of such plants.

### FIELD OF THE INVENTION

The present invention relates to the field of plant breeding and, more specifically, to the development of spinach hybrid variety designated CALLISTO (or NUN 01240 SPS or NUN 01240 or 01240).

### BACKGROUND OF THE INVENTION

The goal of vegetable breeding is to combine various desirable traits in a single variety/hybrid. Such desirable traits may include greater yield, resistance to insects or pests, tolerance to heat and drought, better agronomic quality, higher nutritional value, growth rate and fruit properties.

Breeding techniques take advantage of a plant's method of pollination. There are two general methods of pollination: self-pollination and cross-pollination.

Plants that have been self-pollinated and selected for type over many generations become homozygous at almost all genetic loci and produce a uniform population of true breeding progeny, a homozygous plant. A cross between two such homozygous plants of different lines produces a uniform population of hybrid plants that are heterozygous for many genetic loci.

The development of uniform varieties requires the development of homozygous inbred plants, the crossing of these inbred plants, and the evaluation of the crosses. Pedigree breeding and recurrent selection are examples of breeding methods that have been used to develop inbred plants from breeding populations. Those breeding methods combine the genetic backgrounds from two or more plants or various other broad-based sources into breeding pools from which new lines are developed by selfing and selection of desired phenotypes. The new lines are evaluated to determine which of those have commercial potential.

Spinach (Spinacia oleracea) is a flowering plant in the family Amaranthaceae. Spinach is an annual plant (rarely biennial) having flowers that mature into a small hard dry lumpy fruit cluster about 5-10 mm across containing several seeds.

Spinach has two stages in its life cycle including the vegetative, rosette stage in which the plant is marketable (about 35-40 days from planting) and the bolting, seed stalk stage in which the plant is no longer marketable. Spinach can grow in a range of soils as long as they are moist and fertile, and particularly sandy loams that are high in organic matter.

While breeding efforts to date have provided a number of useful spinach lines with beneficial traits, there remains a great need in the art for new varieties with further improved traits. Such plants would benefit farmers and consumers alike by improving crop yields and/or quality.

### SUMMARY OF THE INVENTION

In one aspect, the present invention provides a seed of CALLISTO a sample of seed of said variety having been deposited under ATCC Accession Number ______.

Another aspect refers to a plant designated CALLISTO, e.g., grown from the seed a sample of seed of said variety having been deposited under ATCC Accession Number ______ or a plant part thereof, such as a seed, pollen, an ovule, a leaf, and a cell.

Also provided are one or more progeny plants (offspring or descendants) of a plant designated CALLISTO obtained by further breeding with said variety designated CALLISTO. Said progeny plant(s) has/have all of the physiological and/or morphological characteristics of variety CALLISTO when grown under the same environmental conditions. In one embodiment, said progeny plant(s) has/have all except one, two or three of the physiological and/or morphological characteristics of CALLISTO. In yet a further embodiment, said progeny retain at least the distinguishing characteristics of CALLISTO.

Moreover, also an Essentially Derived Variety of a spinach plant designated CALLISTO is provided, e.g., an Essentially Derived Variety of CALLISTO having one, two or three physiological and/or morphological characteristics which are different from those of CALLISTO and which otherwise has all the physiological and/or morphological characteristics of CALLISTO when grown under the same environmental conditions of a spinach plant designated CALLISTO. Such an EDV is obtainable by selecting a natural or induced mutant, or a somaclonal variant or natural variant (e.g. an off-type) from a population of plants designated CALLISTO ______ or from a population of progeny of CALLISTO (e.g. an F2, F3 or further selfing population). Such variants are preferably stable, i.e. progeny of such variants retain said one, two or three variant characteristics. Thus, the variant phenotype is preferably genetically stable, also in the mature plants regenerated from the cell or tissue culture. That means, the phenotypic variant does not show variation in phenotype which are transient and are not genetically stable.

Moreover, the present invention refers to a spinach plant, or a part thereof, having all or essentially all the physiological and morphological characteristics of CALLISTO.

Also provided is a tissue culture of regenerable cells of spinach variety CALLISTO, a sample of seed of said variety having been deposited under ATCC Accession Number ______. In one embodiment, said tissue culture comprises cells or protoplasts from a plant part selected from the group consisting of embryos, meristems, cotyledons, pollen, petioles, leaves, anthers, roots, root tips, shoots, pistil, flower, seed and stalks. Also provided is a spinach plant regenerated from said tissue culture, wherein the regenerated plant expresses all, or essentially all, of the physiological and morphological characteristics of spinach variety CALLISTO, a sample of seed of said variety having been deposited under ATCC Accession Number ______.

Another aspect of the invention refers to a method of producing seed, comprising crossing a plant designated CALLISTO with itself or with a second spinach plant and allowing seed to form. In one embodiment, a plant designated CALLISTO is crossed with a spinach plant of a different genotype relative to said plant designated CALLISTO. Thus, also provided is an F1 hybrid seed produced by said method and an F1 hybrid plant produced by growing said F1 hybrid seed. Thus, CALLISTO can be used as a parent plant in a cross with another spinach variety or line.

The invention also refers to a method for producing a seed of a CALLISTO-derived spinach plant (i.e. of progeny or descendants of CALLISTO) comprising the steps of:
(a) crossing a spinach plant designated CALLISTO, a sample of seed of said variety having been deposited under ATCC Accession Number ______, with a second spinach plant; and
(b) allowing seed of a CALLISTO-derived spinach plant to form.

In one embodiment this method further comprises step (c) growing a spinach plant from said seeds.

In one embodiment, this method may further comprise the steps of:
(c) crossing a plant grown from said CALLISTO-derived spinach seed of step (b) with itself or with a second spinach plant to yield additional CALLISTO-derived spinach seed;
(d) growing said additional CALLISTO-derived spinach seed of step (c) to yield additional CALLISTO-derived spinach plants; and optionally
(e) repeating the crossing and growing steps of (c) and (d) one or more times to generate further CALLISTO-derived spinach plants.

Also provided is a method of vegetatively propagating a plant designated CALLISTO comprising the steps of:
(a) collecting cells or tissue capable of being propagated from a plant designated CALLISTO, a sample of seed of said variety having been deposited under ATCC Accession Number ______;
(b) cultivating said cells or tissue to obtain proliferated shoots; and
(c) rooting said proliferated shoots to obtain rooted plantlets.

In one embodiment, this method further comprises growing plants from said rooted plantlets.

Further provided is a method of introducing a desired trait into hybrid variety CALLISTO comprising:
(a) crossing a plant designated CALLISTO, a sample of seed of said variety having been deposited under ATCC Accession Number ______, with a second spinach plant that comprises a desired trait to produce F1 progeny and optionally selfing the F1 progeny to produce F2, F3 or further selfing progeny;
(b) selecting an F1 progeny or F2 or further selfing progeny that comprises the desired trait;
(c) crossing the selected F1, F2 or further selfing progeny with a plant of variety CALLISTO to produce backcross progeny;
(d) selecting backcross progeny comprising the desired trait and the physiological and morphological characteristic of spinach hybrid variety CALLISTO; and
(e) repeating steps (c) and (d) one, two, three or more times in succession to produce higher backcross progeny that comprise the desired trait.

Backcross progeny in step (d) and (e) may also be backcross progeny which are selfed, e.g. BC1S1, BC1S2, etc., BC2S1, BC2S2, etc., BC3S1, etc.

Also provided is a spinach plant produced by this method, wherein the spinach plant comprises essentially all of the physiological and morphological characteristics of spinach hybrid variety CALLISTO when grown under the same conditions, plus the desired trait, a sample of seed of said variety having been deposited under ATCC Accession Number ______. The desired trait may be any trait, such as disease resistance, etc.

Moreover, a method of producing a plant designated CALLISTO, a sample of seed of said variety having been deposited under ATCC Accession Number ______, comprising an added desired trait, the method comprising introducing a transgene conferring the desired trait into a plant of spinach hybrid variety CALLISTO to produce a spinach plant designated CALLISTO comprising an added desired trait.

Also provided is a method of determining the genotype of a plant designated CALLISTO, comprising obtaining a sample of nucleic acids from said plant and detecting in said nucleic acids a plurality of polymorphisms.

In one embodiment, this method further comprises the step of storing the results of the step of detecting the plurality of polymorphisms on a computer readable medium.

Moreover, a method of producing spinach comprising:
(a) obtaining or growing a plant designated CALLISTO, and
(b) collecting leaf tissue from the plant
   is provided herewith.

In still yet another aspect, the invention provides a method of determining the genotype of a plant of spinach variety CALLISTO comprising detecting in the genome of the plant at least a first polymorphism. The method may, in certain embodiments, comprise detecting a plurality of polymorphisms in the genome of the plant. The method may further comprise storing the results of the step of detecting the plurality of polymorphisms on a computer readable medium. The invention further provides a computer readable medium produced by such a method.

These and other features and advantages of this invention are described in, or are apparent from, the following detailed description of various exemplary embodiments of the devices and methods according to this invention.

### FIGURES

FIG. 1 shows cotyledons of CALLISTO and variety PV 0293.

FIG. 2 shows first foliage leaves of CALLISTO and PV 0293.

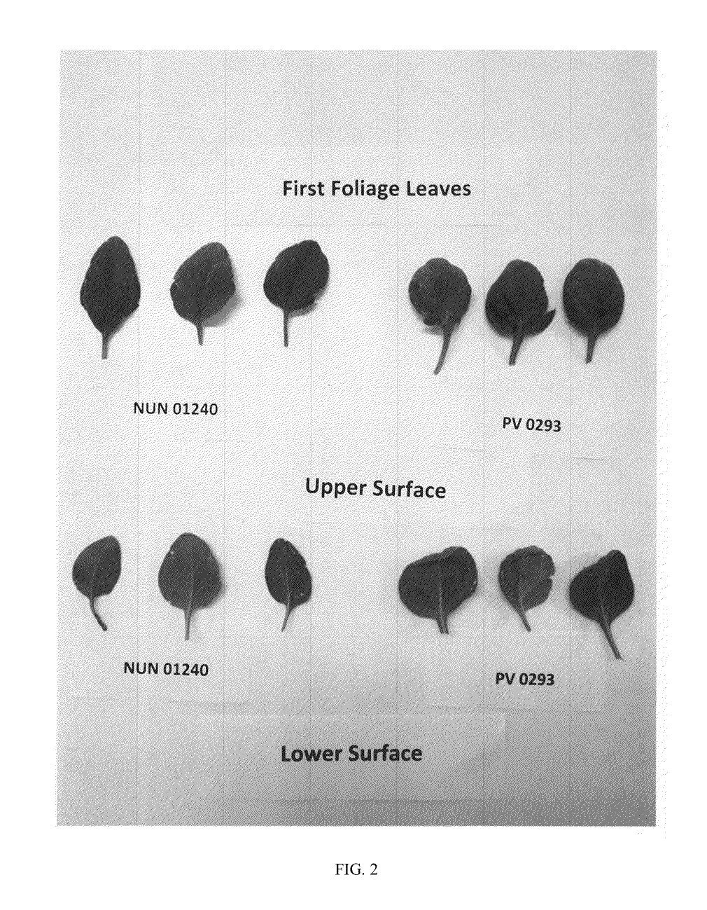

FIG. 3 shows leaves at prime market stage (maturity) of CALLISTO and PV 0293.

### DEFINITIONS

All patent and non-patent literatures are incorporated by reference in their entireties.

In the description and tables herein, a number of terms are used. In order to provide a clear and consistent understanding of the specification and claims, the following definitions are provided:

When used in conjunction with the word “comprising” or other open language in the claims, the words “a” and “an” denote “one or more.”

“Spinach” refers herein to plants of the species Spinacia oleracea L.

“Cultivated spinach” refers to plants of Spinacia oleracea L, i.e. varieties, breeding lines or cultivars of the species Spinacia oleracea L, cultivated by humans and having good agronomic characteristics; preferably such plants are not “wild plants”, i.e. plants which generally have much poorer yields and poorer agronomic characteristics than cultivated plants and e.g. grow naturally in wild populations. “Wild plants” include for example ecotypes, PI (Plant Introduction) lines, landraces or wild accessions or wild relatives of a species.

“USDA descriptors” are the plant variety descriptors described for spinach in the “Objective description of Variety Spinach Spinacia oleracea L.)”, ST-470-83 (as published by U.S. Department of Agriculture, Agricultural Marketing Service, Science and Technology, Plant Variety Protection Office, Beltsville, Md. 20705 (available on the world wide web at www.ams.usda.gov/AMSv1.0/) and which can be downloaded from the world wide web at http://www.ams.usda.gov/AMSv1.0/getfile?dDocName=STELDEV3003749.

“UPOV descriptors” are the plant variety descriptors described for spinach in the “Guidelines for the Conduct of Tests for Distinctness, Uniformity and Stability, TG/85/7 (Geneva 2008), as published by UPOV (International Union for the Protection of New Varieties and Plants, available on the world wide web at upov.int) and which can be downloaded from the world wide web at http://www.upov.int/edocs/tgdocs/en/tg055.pdf and is herein incorporated by reference in its entirety.

“RHS” refers to the Royal Horticultural Society of England which publishes an official botanical color chart quantitatively identifying colors according to a defined numbering system, The chart may be purchased from Royal Horticulture Society Enterprise Ltd RHS Garden; Wisley, Woking; Surrey GU236QB, UK, e.g., the RHS colour chart: 2007 (The Royal Horticultural Society, charity No: 222879, PO Box 313 London SW1P2PE; sold by, e.g., TORSO-VERLAG, Obere Graben 8•D-97877 Wertheim, Article-No.: Art62-00008 EAN-Nr.: 4250193402112).

As used herein, the term “plant” includes the whole plant or any parts or derivatives thereof, preferably having the same genetic makeup as the plant from which it is obtained, such as plant organs (e.g. harvested or non-harvested leaves), plant cells, plant protoplasts, plant cell and/or tissue cultures from which whole plants can be regenerated, plant calli, plant cell clumps, plant transplants, seedlings, hypocotyl, cotyledon, plant cells that are intact in plants, plant clones or micropropagations, or parts of plants (e.g. harvested tissues or organs), such as plant cuttings, vegetative propagations, embryos, pollen, ovules, flowers, leaves, petioles, seeds, clonally propagated plants, roots, stems, root tips, grafts, parts of any of these and the like. Also any developmental stage is included, such as seedlings, cuttings prior or after rooting, mature plants or leaves.

“Harvested plant material” refers herein to plant parts (e.g. a leaf detached from the whole plant) which have been collected for further storage and/or further use.

“Harvested seeds” refers to seeds harvested from a line or variety, e.g. produced after self-fertilization or cross-fertilization and collected.

A plant having “(essentially) all the physiological and/or morphological characteristics” means a plant having essentially all or all the physiological and/or morphological characteristics when grown under the same environmental conditions of the plant of CALLISTO from which it was derived, e.g. the progenitor plant, the parent, the recurrent parent, the plant used for tissue- or cell culture, etc. For example, the plant may have all characteristics mentioned in Table 1. In certain embodiments, the plant having “essentially all the physiological and/or morphological characteristics” are plants having all the physiological and/or morphological characteristics of Table 1, except for certain characteristics, such as one, two or three, mentioned, e.g. the characteristic(s) derived from a converted or introduced gene or trait and/or except for the characteristics which differ in an EDV. So, the plant may have all characteristics mentioned in Table 1, except for one, two or three characteristics of Table 1, in which the plant may thus differ.

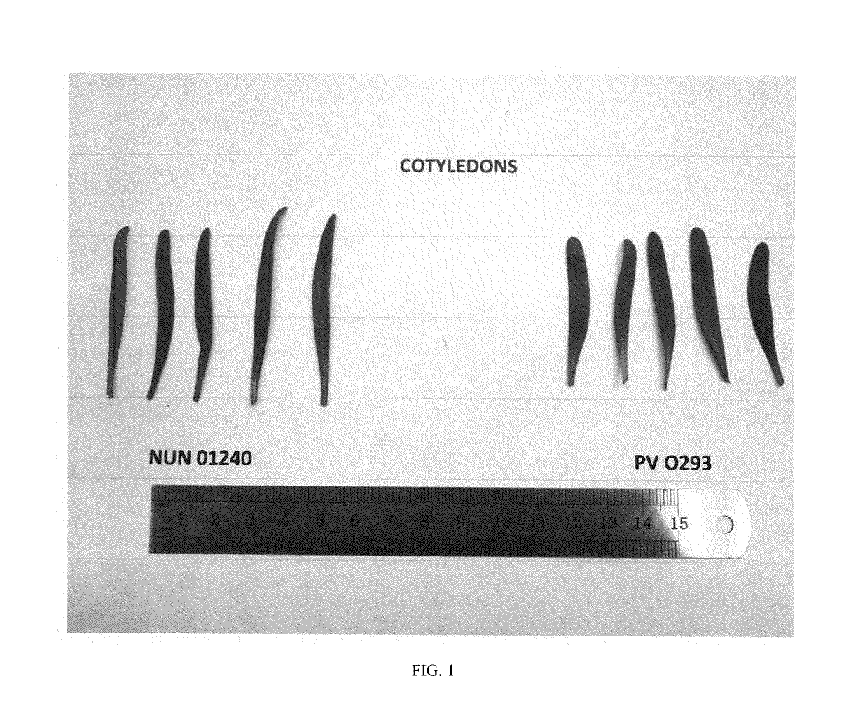

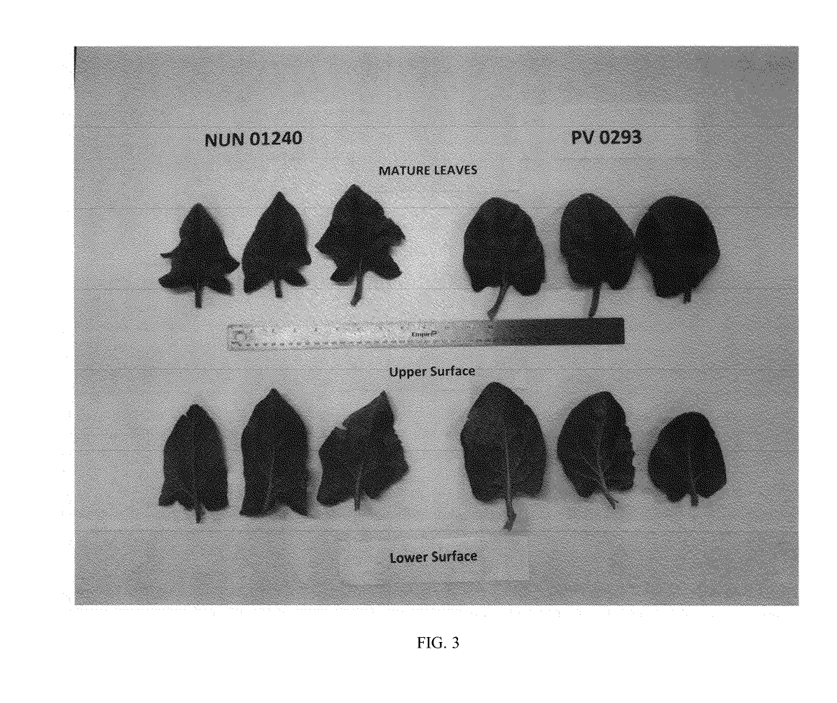

A plant having one or more or all “essential physiological and/or morphological characteristics” or one or more “distinguishing characteristics” (such as one, two, three, four or five) refers to a plant having (or retaining) one or more, or all, or retaining all except one, two or three of the distinguishing characteristics mentioned in Table 1 when grown under the same environmental conditions that distinguish CALLISTO from most similar variety PV0293 such distinguishing characteristics being selected from (but not limited to):
a five-sided shaped leaf (at prime market stage, i.e. mature), whereas PV0293 has circular shaped leaves (USDA descriptor 6. LEAF—Shape); see FIG. 3.
a pointed or round-pointed tip of the leaf (at prime market stage), whereas PV0293 has a rounded tip of the leaf (USDA descriptor 6. LEAF—Tip); see FIG. 3;
a glossy luster of the leaf (at prime market stage) with a darker upper and lower surface color, e.g. RHS Yellow Green 147A and 146A, than PV0293 which has dull leaves with a lighter upper and lower surface color, e.g. RHS Yellow Green 147B and 146B (USDA descriptor 6. LEAF—Tip, Luster and Upper and Lower surface color);
an average petiole length and petiole diameter that is (statistically) significantly longer and significantly narrower than that of PV0293. CALLISTO has a “medium” length petiole that is at least about 25%, or preferably at least 26%, 27%, 28%, 29%, 30%, 31%, 32%, 33%, or even about 33.33% longer than the average petiole length of PV0293. CALLISTO has an average petiole diameter that is at least about 5%, or preferably at least 6%, 6.5%, 7%, 7.5%, or even about 7.8% narrower than the average petiole diameter of PV0293. (USDA descriptor 6. LEAF—Petiole length to the blade and Petiole Diameter);
a leaf (at prime market stage) that has a slightly curled margin (“2=slightly curled”), whereas PV0293 has a curled under margin (“3=curled under”); (USDA descriptor 6. LEAF—Margin).
a (average) plant height (at prime market stage) that is (statistically) significantly taller than that of PV0293; e.g. an average plant height that is at least about 40%, or preferably at least 45%, 50%, 51%, 52%, 53%, 54%, 55%, 56%, 57%, 58%, 59%, or even about 59.7% bigger than the plant height of PV0293; (USDA descriptor 3. PLANT—Size);
A significantly faster growth rate than PV0293; CALLISTO has a growth rate of “3=fast”, while PV0293 has a growth rate of “1=slow” (USDA Descriptor 2. MATURITY—Growth Rate);
An average cotyledon width which is (statistically) significantly narrower than that of PV0293 and an average cotyledon length that is (statistically) significantly longer than that of PV0293; (USDA descriptor 4. SEEDLING COTYLEDON); see FIG. 1;

The physiological and/or morphological characteristics mentioned above are commonly evaluated at significance levels of 1%, 5%, 8% or 10% significance level, when measured under the same environmental conditions. For example, a progeny plant of CALLISTO may have one or more (or all, or all except one, two or three) of the essential physiological and/or morphological characteristics of CALLISTO listed in Table 1, or one or more or all (or all except one, two or three) of the distinguishing characteristics of CALLISTO listed in Table 1 and above, as determined at the 1% or 5% significance level when grown under the same environmental conditions.

As used herein, the term “variety” or “cultivar” means a plant grouping within a single botanical taxon of the lowest known rank, which grouping, irrespective of whether the conditions for the grant of a breeder's right are fully met, can be defined by the expression of the characteristics resulting from a given genotype or combination of genotypes, distinguished from any other plant grouping by the expression of at least one of the said characteristics and considered as a unit with regard to its suitability for being propagated unchanged.

A variety is referred to as an “Essentially Derived Variety” (EDV) i.e., shall be deemed to be essentially derived from another variety, “the initial variety” when (i) it is predominantly derived from the initial variety, or from a variety that is itself predominantly derived from the initial variety, while retaining the expression of the essential characteristics that result from the genotype or combination of genotypes of the initial variety; (ii) it is clearly distinguishable from the initial variety; and (iii) except for the differences which result from the act of derivation, it conforms to the initial variety in the expression of the essential characteristics that result from the genotype or combination of genotypes of the initial variety. Thus, an EDV may be obtained for example by the selection of a natural or induced mutant, or of a somaclonal variant, or of an off-type, the selection of a variant individual from plants of the initial variety or from progeny of the initial variety (e.g. F1, F2, etc.), backcrossing, or transformation by genetic engineering.

“Plant line” is for example a breeding line which can be used to develop one or more varieties.

“Hybrid variety” or “F1 hybrid” refers to the seeds harvested from crossing two inbred (nearly homozygous) parental lines. For example, the female parent is pollinated with pollen of the male parent to produce hybrid (F1) seeds on the female parent.

“Regeneration” refers to the development of a plant from cell culture or tissue culture or vegetative propagation.

“Vegetative propagation”, “vegetative reproduction” or “clonal propagation” are used interchangeably herein and mean the method of taking part of a plant and allowing that plant part to form at least roots where plant part is, e.g., defined as or derived from (e.g. by cutting of) leaf, pollen, embryo, cotyledon, petiole, hypocotyl, cells, protoplasts, meristematic cell, root, root tip, pistil, anther, flower, shoot tip, shoot, stem, etc. When a whole plant is regenerated by vegetative propagation, it is also referred to as a vegetative propagation or a vegetatively propagated plant.

“Selfing” refers to self-pollination of a plant, i.e., the transfer of pollen from the anther to the stigma of the same plant. “Crossing” refers to the mating of two parent plants.

“Average” refers herein to the arithmetic mean.

“Locus” (plural loci) refers to the specific location of a gene or DNA sequence on a chromosome. A locus may confer a specific trait.

“Allele” refers to one or more alternative forms of a gene locus. All of these loci relate to one trait. Sometimes, different alleles can result in different observable phenotypic traits, such as different pigmentation. However, many variations at the genetic level result in little or no observable variation. If a multicellular organism has two sets of chromosomes, i.e. diploid, these chromosomes are referred to as homologous chromosomes. Diploid organisms have one copy of each gene (and therefore one allele) on each chromosome. If both alleles are the same, they are homozygotes. If the alleles are different, they are heterozygotes.

“Genotype” refers to the genetic composition of a cell or organism.

The term “traditional breeding techniques” encompasses herein crossing, selfing, selection, double haploid production, embryo rescue, protoplast fusion, marker assisted selection, mutation breeding etc. as known to the breeder (i.e. methods other than genetic modification/transformation/transgenic methods), by which, for example, a genetically heritable trait can be transferred from one spinach line or variety to another.

“Backcrossing” is a traditional breeding technique used to introduce a trait into a plant line or variety. The plant containing the trait is called the donor plant and the plant into which the trait is transferred is called the recurrent parent. An intial cross is made between the donor parent and the recurrent parent to produce progeny plants. Progeny plants which have the trait are then crossed to the recurrent parent. After several generations of backcros sing and/or selfing the recurrent parent comprises the trait of the donor. The plant generated in this way may be referred to as a “single trait converted plant”.

“Progeny” or “descendants” as used herein refers to plants derived from a plant designated CALLISTO. Progeny may be derived by regeneration of cell culture or tissue culture or parts of a plant designated CALLISTO or selfing of a plant designated CALLISTO or by producing seeds of a plant designated CALLISTO. In further embodiments, progeny may also encompass plants derived from crossing of at least one plant designated CALLISTO with another spinach plant of the same or another variety or (breeding) line, or with a wild spinach plant, backcrossing, inserting of a locus into a plant or selecting a plant comprising a mutation or selecting a variant. A progeny is, e.g., a first generation progeny, i.e. the progeny is directly derived from, obtained from, obtainable from or derivable from the parent plant by, e.g., traditional breeding methods (selfing and/or crossing) or regeneration. However, the term “progeny” generally encompasses further generations such as second, third, fourth, fifth, sixth, seventh or more generations, i.e., generations of plants which are derived from, obtained from, obtainable from or derivable from the former generation by, e.g., traditional breeding methods, regeneration or genetic transformation techniques. For example, a second generation progeny can be produced from a first generation progeny by any of the methods mentioned above. Especially progeny of CALLISTO which are EDVs or which retain all (or all except 1, 2 or 3) physiological and/or morphological characteristics of CALLISTO listed in Table 1, or which retain all (or all except 1, 2, or 3) of the distinguishing characteristics of CALLISTO described elsewhere herein and in Table 1, are encompassed herein.

“Tissue Culture” or “cell culture” refers to a in vitro composition comprising isolated cells of the same or a different type or a collection of such cells organized into parts of a plant.

The terms “gene converted” or “conversion plant” in this context refer to spinach plants which are developed by backcrossing wherein essentially all of the desired morphological and/or physiological characteristics of parent are recovered in addition to the one or more genes transferred into the parent via the backcrossing technique or via genetic engineering. Likewise a “Single Locus Converted (Conversion) Plant” refers to plants which are developed by plant breeding techniques comprising or consisting of backcrossing, wherein essentially all of the desired morphological and/or physiological characteristics of a spinach variety are recovered in addition to the characteristics of the single locus having been transferred into the variety via the backcrossing technique and/or by genetic transformation.

“Transgene” or “chimeric gene” refers to a genetic locus comprising a DNA sequence which has been introduced into the genome of a spinach plant by transformation. A plant comprising a transgene stably integrated into its genome is referred to as “transgenic plant”.

“Haploid” refers to a cell or organism having one set of the two sets of chromosomes in a diploid.

“Diploid” refers to a cell or organism having two sets of chromosomes.

“Triploid” refers to a cell or organism having three sets of chromosomes.

“Tetraploid” refers to a cell or organism having four sets of chromosomes.

The term “mean” refers to the arithmetic mean of several measurements. The skilled person understands that the appearance of a plant depends to some extent on the growing conditions of said plant. Thus, the skilled person will know typical growing conditions for spinach described herein. The mean, if not indicated otherwise within this application, refers to the arithmetic mean of measurements on at least 10 different, randomly selected plants or plant parts of a variety or line.

The term “substantially equivalent” or “not significantly different” refers to a characteristic that, when compared, does not show a statistically significant difference (e.g., p≧0.05 using ANOVA) from the mean. Vice versa, “significantly different” or “statistically significantly different” refers to a characteristic that, when compared, does show a statistically significant difference (e.g., p<0.05 using ANOVA) from the mean.

### DETAILED DESCRIPTION OF THE INVENTION

A number of characteristics are important to spinach breeders including (but not limited to) disease resistance, earliness/start of bolting (early, medium, late), growth habit and leaf characteristics.

The present invention relates to a Spinacia oleracea L. hybrid variety, referred to as CALLISTO, or progeny, or EDVs thereof, which has an ovate shape of first foliage leaves with a slightly curled margin and a round-pointed tip and which has a five-sided shape of leaf at maturity (prime market stage) with pointed or round-pointed tips and a slightly curled margin. CALLISTO is considered the most similar to PV 0293, a commercially available variety of Pop Vriend Seeds.

CALLISTO can be easily distinguished from PV 0293 when grown under the same environmental conditions.

Variety CALLISTO provided herein differs from the most similar comparison variety PV 0293, when grown under the same environmental conditions, in one or more characteristics (referred herein to as “distinguishing characteristics” or “distinguishing morphological and/or physiological characteristics” (or essential physiological and/or morphological characteristics) selected from:
a five-sided shaped leaf (at prime market stage, i.e. mature), whereas PV0293 has circular shaped leaves (USDA descriptor 6. LEAF—Shape); see FIG. 3.
a pointed or round-pointed tip of the leaf (at prime market stage), whereas PV0293 has a rounded tip of the leaf (USDA descriptor 6. LEAF—Tip); see FIG. 3;
a glossy luster of the leaf (at prime market stage) with a darker upper and lower surface color, e.g. RHS Yellow Green 147A and 146A, than PV0293 which has dull leaves with a lighter upper and lower surface color, e.g. RHS Yellow Green 147B and 146B (USDA descriptor 6. LEAF—Tip, Luster and Upper and Lower surface color);
an average petiole length and petiole diameter that is (statistically) significantly longer and significantly narrower than that of PV0293. CALLISTO has a “medium” length petiole that is at least about 25%, or preferably at least 26%, 27%, 28%, 29%, 30%, 31%, 32%, 33%, or even about 33.33% longer than the average petiole length of PV0293. CALLISTO has an average petiole diameter that is at least about 5%, or preferably at least 6%, 6.5%, 7%, 7.5%, or even about 7.8% narrower than the average petiole diameter of PV0293. (USDA descriptor 6. LEAF—Petiole length to the blade and Petiole Diameter);
a leaf (at prime market stage) that has a slightly curled margin (“2=slightly curled”), whereas PV0293 has a curled under margin (“3=curled under”); (USDA descriptor 6. LEAF—Margin).
a (average) plant height (at prime market stage) that is (statistically) significantly taller than that of PV0293; e.g. an average plant height that is at least about 40%, or preferably at least 45%, 50%, 51%, 52%, 53%, 54%, 55%, 56%, 57%, 58%, 59%, or even about 59.7% bigger than the plant height of PV0293; (USDA descriptor 3. PLANT—Size);
A significantly faster growth rate than PV0293; CALLISTO has a growth rate of “3=fast”, while PV0293 has a growth rate of “1=slow” (USDA Descriptor 2. MATURITY—Growth Rate);
An average cotyledon width which is (statistically) significantly narrower than that of PV0293 and an average cotyledon length that is (statistically) significantly longer than that of PV0293; (USDA descriptor 4. SEEDLING COTYLEDON); see FIG. 1;

It is understood that “significant” differences refer to statistically significant differences, when comparing the characteristic between two plant lines or varieties when grown under the same conditions. Preferably at least about 10, 15, 20, 30, 40, 50 or more plants per line or variety are grown under the same conditions and characteristics are measured on at least about 10, 15, 20 or more randomly selected plant or plant parts to obtain averages. Thus, physiological and/or morphological characteristics or traits are commonly evaluated at a significance level of 1%, 5%, 8% or 10%, when measured in plants grown under the same environmental conditions. For example, two replications of 50 plants each, from which 15 or more plants or plant parts were randomly selected can be used to measure characteristics.

In one aspect, the invention provides seeds of the spinach hybrid variety designated CALLISTO wherein a representative sample of seeds of said variety was deposited under the Budapest Treaty, with Accession number NCIMB ______.

In one specific aspect of the invention a spinach plant is provided, which does not (statistically significantly) differ from CALLISTO in any of the morphological and/or physiological characteristics of Table 1, but which further comprises an added trait.

In one aspect a spinach plant is provided, which (statistically significantly) differs from CALLISTO in at least one, two or three of the morphological and/or physiological characteristics of Table 1, but which otherwise does not significantly differ from CALLISTO and any of the other morphological and/or physiological characteristics of Table 1, and which plant further comprises an added trait.

In one aspect a spinach plant is provided, which (statistically significantly) differs from CALLISTO in at least one, two or three of the morphological and/or physiological characteristics of Table 1, but which otherwise does not significantly differ from CALLISTO and any of the other morphological and/or physiological characteristics of Table 1.

In another aspect, a spinach plant is provided, which (statistically significant) differs from CALLISTO in at least one morphological and/or physiological characteristic, but which does not differ from CALLISTO in at least 5, 6, 7 or all 8 of the following (distinguishing) characteristics when grown under the same conditions: 1) shape of the leaf at prime market stage; 2) tip of the leaf at prime market stage; 3) color and luster of leaf at prime market stage; 4) average petiole length and diameter; 5) curling of the margin of the mature leaf; 6) average plant height at prime market stage; 7) growth rate; 8) average cotyledon length and width;

In a further aspect, a spinach plant, which (statistically significant) differs from CALLISTO in at least one morphological and/or physiological characteristics, but which does not differ from CALLISTO in at least 5, 6, 7, 8, 9 or all 10 of the following characteristics when grown under the same conditions: 1) shape of the leaf at prime market stage; 2) tip of the leaf at prime market stage; 3) color and luster of leaf at prime market stage; 4) average petiole length and diameter; 5) curling of the margin of the mature leaf; 6) average plant height at prime market stage; 7) growth rate; 8) average cotyledon length and width; 9) a semi-erect plant habit at maturity; 9) a smooth leaf surface at maturity; 10) resistance against Downey Mildew (P. spinaceae) races 1-14.

In one embodiment the characteristics of any of the above spinach plants have/are (where mentioned above): 1) a five-sided shape of the leaves at prime market stage; 2) a pointed tip or round-pointed tip of the leaves at prime market stage; 3) a light green upper and lower surface color of the mature leaves, e.g. RHS Yellow Green 147A and RHS Yellow Green 146A, and a glossy luster of the leaves at prime market stage; 4) a average petiole length of at least 3.5, 3.8, 4.0, 4.2 or 4.4 cm and an average petiole diameter of equal to or less than 6.2 mm, e.g. equal to or less than 6.0 mm, 5.9 mm or 5.8 mm; 5) a slightly curled leaf margin; 6) an average plant height at prime market stage that is at least about 9.0 cm, or preferably at least about 10 cm, 11 cm, 12 cm, 13 cm, or even about 13.1 cm; 7) a fast growth rate (selected from the categories slow (similar to Norgreen), medium (similar to Long Standing Bloomsdale) and fast (similar to Dixie Market); 8) an average cotyledon length of at least about 45.0 mm, preferably at least about 46.0 mm, 47.0 mm, 48.0 mm, 49.0 mm or even 49.2 mm; and an average cotyledon width of less than 5.0 mm, e.g. equal to or less than 4.9 mm, 4.8 mm, 4.7 mm, or 4.6 mm; 9) a smooth leaf surface at maturity; and 10) resistance against Downey Mildew (P. spinaceae) races 1-14.

Seeds of CALLISTO, i.e. seeds from which a plant of the variety CALLISTO can be grown, are obtainable by crossing the male parent with the female parent and harvesting the seeds produced on the female parent. The resultant CALLISTO seeds, of which a representative sample has been deposited under Accession Number NCIMB ______, can be grown to produce CALLISTO plants. In one embodiment a plurality of CALLISTO seeds are packaged into small and/or large containers (e.g., bags, cartons, cans, etc.). The seeds may be primed and/or treated with various compounds, such as seed coatings and/or fungicides and/or insecticides.

Also provided are plants of spinach hybrid variety CALLISTO produced from growing seeds of variety CALLISTO or vegetative propagations of variety CALLISTO, wherein a representative sample of said seeds has been deposited under the Budapest Treaty, with Accession Number NCIMB ______. For example, plants of CALLISTO can be produced by seeding directly in the ground (e.g., field) or by germinating the seeds in controlled environment conditions (e.g., greenhouses) and then transplanting the seedlings into the field.

In accordance with one aspect of the present invention, there is provided a plant having all the physiological and/or morphological characteristics of CALLISTO. A description of the physiological and/or morphological characteristics of spinach variety CALLISTO is presented, e.g., in Table 1.

Also provided are variants of variety CALLISTO, such as natural or induced mutants, somaclonal variants, or a natural variant (e.g. off-type) selected from a plurality of CALLISTO plants or from descendants of CALLISTO. In one aspect the variant has all the physiological and/or morphological characteristics of CALLISTO of Table 1, except one, two or three, in which the variant differs (genetically stably). In another aspect the variant has at least all the distinguishing characteristics (numbered 1-8 above) of variety CALLISTO. In yet another aspect the variant has at least all the distinguishing characteristics (numbered 1-8 above) of variety CALLISTO and has further the characteristics 9) a smooth leaf surface at maturity; 10) resistance against Downey Mildew (P. spinaceae) races 1-14.

In other aspects, the invention provides for progeny (or descendants) of variety CALLISTO such as progeny obtained by further breeding with CALLISTO. Further breeding with CALLISTO includes selfing CALLISTO one or more times and/or cross-pollinating CALLISTO with another spinach plant or variety one or more times.

In particular, the invention provides for progeny that retain all or all except one, two or three of the essential morphological and/or physiological characteristics of CALLISTO or that retain one or more or all or all except one, two or three of the distinguishing characteristics of the spinach type described further above and when grown under the same environmental conditions as CALLISTO.

In another aspect, the invention provides for vegetative reproductions of the variety CALLISTO, or of progeny thereof, and the invention also provided for essentially derived varieties (EDVs) of CALLISTO, or of progeny thereof, and vegetative reproductions of such EDVs.

The morphological and/or physiological differences between plants according to the invention, i.e. CALLISTO or progeny thereof, or an EDV thereof, and other known varieties can easily be established by growing CALLISTO (or progeny or EDVs) next to the other varieties (in the same field, under the same environmental conditions), preferably in several locations which are suitable for said spinach cultivation, and measuring morphological and/or physiological characteristics of a number of plants (e.g., to calculate an average value and to determine the variation range/uniformity within the variety). For example, trials can be carried out in California, e.g. in or near Acampo Calif., USA (N 38 degrees 07′261″/W 121 degrees 18′ 807″, USA), whereby e.g., days from planting to prime market stage; plant habit; plant size; width, length, tip and color of seedling cotyledons; shape, base, tip, margin, upper and lower surface color of first foliage leaves; surface, shape, base, tip, margin, upper and lower surface color, luster, blade size, blade lobing, petiole color, petiole red pigmentation, petiole length and petiole diameter of leaves at prime market stage; seed stalk development; seed surface; disease resistances and/or insect resistances can be measured and directly compared (see USDA descriptors).

Morphological and/or physiological characteristics (and distinguishing characteristics which distinguish CALLISTO from PV 0293) of CALLISTO, are provided in the Examples, in Table 1. Encompassed herein are also plants derivable from CALLISTO (e.g. by selfings and/or crossing and/or backcrossing with CALLISTO and/or mutant selection and/or natural variant selection) comprising all (or all except one, two or three) of the physiological and/or morphological characteristics of CALLISTO listed in Table 1 as determined at the 5% significance level when grown under the same environmental conditions and/or comprising one or more (or all; or all except one, two or three) of the distinguishing characteristics as determined at the 5% significance level when grown under the same environmental conditions. The skilled person understands that the morphological and/or physiological characteristics of a plant may vary somewhat with variation in the environment (such as temperature, light intensity, day length, humidity, soil, fertilizer use), which is why a comparison under the same environmental conditions is preferred. Colors can best be measured against The Munsell Book of Color (Munsell Color Macbeth Division of Kollmorgan Instruments Corporation) or using the Royal Horticultural Society Chart (http://www.rhs.org.uk/Plants/RHS-Publications/RHS-colour-charts).

Also at-harvest and/or post-harvest characteristics of leaves can be compared. For example, cold storage holding quality (e.g., yellowing) can be measured using known methods.

An EDV according to the invention is an Essentially Derived Variety of CALLISTO having one, two or three physiological and/or morphological characteristics which are different from those of CALLISTO and which otherwise has all the physiological and morphological characteristics of CALLISTO, wherein a representative sample of seed of variety CALLISTO has been deposited under Accession Number NCIMB ______. In particular, variants which differ from CALLISTO in only one, two or three of the characteristics mentioned in Table 1 are encompassed.

In one aspect, the EDV differs from CALLISTO in one, two or three of the distinguishing morphological and/or physiological characteristics selected from: 1) shape of the leaf at prime market stage; 2) tip of the leaf at prime market stage; 3) color and luster of leaf at prime market stage; 4) average petiole length and diameter; 5) curling of the margin of the mature leaf; 6) average plant height at prime market stage; 7) growth rate; 8) average cotyledon length and width. In another embodiment the EDV may differ from CALLISTO in one, two or three morphological and/or physiological characteristic of Table 1 other than the “distinguishing morphological and/or physiological characteristics” 1) to 8) above (or essential physiological and/or morphological characteristics) of CALLISTO for example selected from (but not limited to): the shape of the cotyledon tip; cotyledon color; mature leaf surface; leaf blade lobing, petiole color, petiole red pigmentation, start of bolting, seed surface or others.

In still yet another aspect of the invention, the genetic complement of CALLISTO is provided. The phrase “genetic complement” is used to refer to the aggregate of nucleotide sequences, the expression of which sequences defines the phenotype of, in the present case, a spinach plant, or a cell or tissue of that plant. A genetic complement thus represents the genetic make-up of a cell, tissue or plant, and a hybrid genetic complement represents the genetic make-up of a hybrid cell, tissue or plant.

The invention thus provides spinach plant cells that have a genetic complement in accordance with the spinach plant cells disclosed herein, and plants, seeds and plants containing such cells. Plant genetic complements may be assessed by genetic marker profiles, and by the expression of phenotypic traits that are characteristic of the expression of the genetic complement, e.g., isozyme typing profiles. It is understood that CALLISTO could be identified by any of the many well-known techniques such as, for example, Simple Sequence Length Polymorphisms (SSLPs), Randomly Amplified Polymorphic DNAs (RAPDs), DNA Amplification Fingerprinting (DAF), Sequence Characterized Amplified Regions (SCARs), Arbitrary Primed Polymerase Chain Reaction (AP-PCR), Amplified Fragment Length Polymorphisms (AFLPs) (see, e.g., EP 534 858), and Single Nucleotide Polymorphisms (SNPs).

In other aspects, the invention provides for a plant part of CALLISTO or of a progeny of CALLISTO (such as a plant having all the physiological and morphological characteristics of CALLISTO or a hybrid of CALLISTO), or a derived variety of CALLISTO, e.g., an EDV or variant of CALLISTO, (such as a plant having all except one, two or three of the morphological and physiological characteristics of CALLISTO). Parts of CALLISTO (or from its progeny or from an EDV of CALLISTO) encompass any cells, tissues, organs obtainable from the seedlings or plants, such as but not limited to: spinach leaves or parts thereof, cuttings, hypocotyl, petiole, cotyledon, pollen, flowers, anthers, embryos, ovaries, and the like. Preferably, a part of a plant part of CALLISTO or its progeny or an EDV of CALLISTO is a leaf, pollen, flowers, shoot or cutting or parts thereof. Such parts can be stored and/or processed further. Encompassed are therefore also food or feed products comprising one or more of such parts, such as fresh, dried, frozen spinach leaves or parts thereof, etc. from CALLISTO or from progeny thereof, or from a derived variety, such as an EDV. In another embodiment, the invention provides for a container comprising or consisting of a plurality of harvested spinach leaves of CALLISTO, or progeny thereof, or a derived variety, such as an EDV. For example, containers such as cans, boxes, crates, bags, cartons, Modified Atmosphere Packagings, films (e.g. biodegradable films), etc. comprising plant parts of plants (fresh and/or processed) described herein are also provided herein.

The invention also provides a spinach plant comprising at least a first set of the chromosomes of spinach variety CALLISTO, a sample of seed having been deposited under Accession Number NCIMB ______; further provided is a spinach plant comprising a single locus conversion, wherein said spinach plant has essentially all of the morphological and/or physiological characteristics of the spinach plant comprising at least a first set of the chromosomes of spinach variety CALLISTO. In another embodiment, this single locus conversion confers a trait selected from the group consisting of male sterility, herbicide tolerance, insect resistance, pest resistance, disease resistance, environmental stress tolerance, modified carbohydrate metabolism and modified protein metabolism.

In one embodiment, CALLISTO may also be mutated (by e.g. irradiation, chemical mutagenesis, heat treatment, etc.) and mutated seeds or plants may be selected in order to change one or more characteristics of CALLISTO, e.g. one, two, three or more of the essential physiological and/or morphological characteristics of CALLISTO may be changed. The selected plant is a variant of CALLISTO. The one, two, three or more changed characteristics may be one, two or three of the distinguishing characteristics of CALLISTO or one, two or three of the other characteristics of Table 1 (not the distinguishing characteristics). Also natural mutants or natural variants of CALLISTO may be identified and used in breeding. For example, somaclonal variants can be selected, which are genetically stable, or a natural variant can be selected by sowing seeds of CALLISTO and selecting a variant (or off-type) from those seedlings or plants. Methods such as TILLING and/or EcoTILLING may be applied to spinach populations in order to identify mutants. Similarly, CALLISTO may be transformed and regenerated, whereby one or more chimeric genes are introduced into the variety or progeny thereof or into an EDV thereof. Transformation can be carried out using standard methods, such as Agrobacterium tumefaciens mediated transformation or biolistics, followed by selection of the transformed cells and regeneration into plants. A desired trait (e.g. genes conferring pest or disease resistance, herbicide, fungicide or insecticide tolerance, etc.) can be introduced into CALLISTO, or progeny thereof, by transforming CALLISTO or progeny thereof with a transgene that confers the desired trait, wherein the transformed plant retains all the phenotypic and/or morphological and/or physiological characteristics of CALLISTO or the progeny thereof and contains the desired trait conferred by the chimeric gene. Thus, also a transgenic spinach plant comprising a transgene conferring a desired trait and further comprising all (or all except one two or three) of the morphological and/or physiological characteristics of CALLISTO, or further comprising all (or all except one, two or three) of the distinguishing characteristics of CALLISTO, is provided herein.

In one aspect, haploid plants and/or double haploid plants of CALLISTO, or an EDV of CALLISTO, or progeny of any of these, are encompassed herein. Haploid and double haploid (DH) plants can, for example, be produced by cell or tissue culture, optionally treatment with chromosome doubling agents, and regeneration into a whole plant. For DH production chromosome doubling may be induced using known methods, such as colchicine treatment or the like.

The invention also provides for a method of producing a new spinach plant. The method comprises crossing a plant of the invention CALLISTO, either as male or as female parent, with a second spinach plant (or a wild relative of spinach) one or more times, and/or selfing a spinach plant CALLISTO one or more times and optionally selecting progeny from said crossing and/or selfing. In one embodiment, the said second spinach plant is a Spinacia oleracea L plant, e.g. a cultivated S. oleracea, especially a breeding line or variety. Thus, invention also provides a method for developing a spinach plant in a spinach breeding program, using a spinach plant of the invention or a part thereof as a source of plant breeding material. Suitable plant breeding techniques are recurrent selection, backcrossing, pedigree breeding, mass selection, mutation breeding and/or genetic marker enhanced selection.

In one embodiment, progeny of a plant according to the invention are either the generation (seeds) produced from the first cross (F1) or selfing (S1), or any further generation produced by crossing and/or selfing (F2, F3, etc.) and/or backcrossing (BC1, BC2, BC1S1, BC1S2, etc.) one or more selected plants of the F1 and/or S1 and/or BC1 generation (or plants of any further generation, e.g. the F2) with another spinach plant (and/or with a wild relative of spinach). Progeny may have all (or all except one, two or three) of the physiological and/or morphological characteristics of spinach variety CALLISTO when grown under the same environmental conditions and/or progeny may have (be selected for having) one or more, or all or all except one, two or three of the distinguishing characteristics of spinach of the invention. Using common breeding methods such as backcrossing or recurrent selection, one or more specific characteristics may be introduced into CALLISTO.

One embodiment of the present invention refers to spinach seeds and plants produced by a process that comprises crossing a first parent spinach plant with a second parent spinach plant, wherein at least one of the first or second parent spinach plants is a plant of the variety designated CALLISTO. In one preferred embodiment of the invention, spinach seed and plants produced by the process are first generation (F1) hybrid spinach seed and plants produced by crossing a plant in accordance with the invention with another, distinct plant. The present invention further contemplates plant parts, especially leaves, of such an F1 hybrid spinach plant, and methods of use thereof.

The development of new varieties using one or more starting varieties is well known in the art. In accordance with the present invention, processes are provided for producing spinach seeds and plants which processes generally comprise crossing a first parent spinach plant with a second parent spinach plant, wherein at least one of the first or second parent spinach plants is a plant of the variety designated CALLISTO. Thus, in accordance with the invention, novel varieties may be created by crossing CALLISTO optionally followed by multiple generations of breeding according to such well known methods. These processes may be further exemplified as processes for preparing hybrid spinach seed or plants comprising crossing a plant designated CALLISTO with a second spinach plant one or more times, and selecting progeny from said crossing. In these processes, crossing will result in the production of seed. The seed production occurs regardless of whether the seed is collected or not.

New lines or varieties may be created by crossing CALLISTO with any second spinach plant. In selecting such a second plant to cross for the purpose of developing novel lines, it may be desired to choose those plants which either themselves exhibit one or more selected desirable characteristics or which exhibit the desired characteristic(s) in progeny. Once initial crosses have been made, inbreeding and selection take place to produce new varieties. For development of a uniform line, often five or more generations of selfing and selection are involved.

In one embodiment of the invention, the first step in “crossing” comprises planting seeds of a first and second parent spinach plant, often in proximity so that pollination will occur for example, mediated by insect vectors. Alternatively, pollen can be transferred manually. Where the plant is self-pollinated, pollination may occur without the need for direct human intervention other than plant cultivation.

A second step may comprise cultivating or growing the seeds of first and second parent spinach plants into plants that bear flowers. A third step may comprise preventing self-pollination of the plants, such as by emasculating the male portions of flowers, (i.e., treating or manipulating the flowers to produce an emasculated parent spinach plant). Self-incompatibility systems may also be used in some hybrid crops for the same purpose. Self-incompatible plants still shed viable pollen and can pollinate plants of other varieties but are incapable of pollinating themselves or other plants of the same line.

A fourth step for a hybrid cross may comprise cross-pollination between the first and second parent spinach plants. Yet another step comprises harvesting the seeds from at least one of the parent spinach plants. The harvested seed can be grown to produce a spinach plant or hybrid spinach plant.

Backcrossing can also be used to improve an inbred plant or a variety. Backcrossing transfers a specific desirable trait from one inbred or non-inbred source to an inbred plant or variety that lacks that trait.

In a typical backcross protocol, the original line or variety of interest (recurrent parent) is crossed to a second line or variety (non-recurrent parent) that carries the single locus of interest to be transferred. The resulting progeny from this cross, e.g. a selected F1, F2 or F3 individual, are then crossed again to the recurrent parent and the process is repeated until a spinach plant is obtained wherein essentially all of the desired morphological and/or physiological characteristics of the recurrent parent are recovered in the converted plant, in addition to the single transferred locus from the non-recurrent parent. The selection of a suitable recurrent parent is an important step for a successful backcrossing procedure. The goal of a backcross protocol is to alter or substitute a single trait or characteristic in the original line or variety. To accomplish this, a single locus of the recurrent variety is modified or substituted with the desired locus from the non-recurrent parent, while retaining essentially all of the rest of the desired genetic, and therefore the desired physiological and/or morphological constitution of the original line or variety. The choice of the particular non-recurrent parent will depend on the purpose of the backcross; one of the major purposes is to add some commercially desirable trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

In one embodiment, progeny diploid spinach plants of a backcross in which CALLISTO is the recurrent parent comprise (i) the desired trait from the non-recurrent parent and (ii) all of the physiological and morphological characteristics of CALLISTO as determined at the 5% significance level when grown in the same environmental conditions.

Spinach varieties can also be developed from more than two parents. The technique, known as modified backcrossing, uses different recurrent parents during the backcrossing. Modified backcrossing may be used to replace the original recurrent parent with a variety having certain more desirable characteristics or multiple parents may be used to obtain different desirable characteristics from each. Many single locus traits have been identified that are not regularly selected for in the development of a new inbred but that can be improved by backcrossing techniques. Single locus traits mayor may not be transgenic; examples of these traits include, but are not limited to, male sterility, herbicide resistance, resistance to bacterial, fungal, or viral disease, insect resistance, restoration of male fertility, modified fatty acid or carbohydrate metabolism, and enhanced nutritional quality. These comprise genes generally inherited through the nucleus.

Examples of desirable characteristics may include, but are not limited to, resistance to one or more of the following diseases Peronospora farinose f. sp. spinaciae, e.g. to new races and/or race 510C, race UA4612, race UA4410 or others; white rust (Albugo occidentalis), Fusarium oxysporum f. sp. spinaciae, Pythium resistance, Rhizoctonia resistance, Colletotrichum anthracnose resistance, Cercospora beticola resistance, Verticillium dahliae resistance, Phytophthora ssp resistance, Stemphylium leaf spot resistance, Curly Top Virus resistance, Cucumber Mosaic Virus (CMV) resistance, Impatiens Necrotic Spot Virus (INSV), Beet Yellows and/or Beet mosaic resistance, leaf miner resistance, and others.

Non-limiting examples of genes that may be utilized for generating transgenic spinach include RAR1 disease resistance proteins (see, e.g., U.S. Pat. No. 7,098,378, the ability to tolerate high salt conditions, as described in (see, e.g., U.S. Pat. No. 7,041,875); trehalose synthase for increased amounts of trehalose to increase tolerance to a variety of stresses, in particular to decreased availability of water (see, e.g., U.S. Pat. No. 5,792,92); overexpression of phytochrome, such as for increased shade tolerance and/or darker green color, as described in, for example, U.S. Pat. No. 5,268,526; expression of reversibly glycosylated protein (RGP) for at least altered growth rates, as described in, for example, U.S. Pat. No. 6,194,638; improved growth under low-light conditions (see, e.g., U.S. Pat. No. 7,081,363).

One aspect of the present invention refers to a method of introducing a single locus conversion, or single trait conversion, into a spinach plant according to the invention and/or into CALLISTO comprising:

(a) crossing a spinach plant of variety CALLISTO, a representative sample of seed of said variety having been deposited under Accession Number NCIMB ______, with a second spinach plant comprising a desired single locus to produce F1 progeny plants and optionally selfing the F1 one or more times to produce F2 or further generation selfings;
(b) selecting an F1, F2 or further generation selfing progeny plant that have the single locus or trait to produce a selected progeny plant;
(c) crossing the selected progeny plant with a plant of CALLISTO, to produce backcross progeny plants;
(d) selecting backcross progeny plants that have the single locus (or trait) and one or more or all (or all except one, two or three) of the distinguishing characteristics of the spinach according to the invention and/or all (or all except one, two or three) of the physiological and/or morphological characteristics of CALLISTO to produce selected backcross progeny plants; and
(e) repeating steps (c) and (d) one or more times in succession to produce selected second, third, fourth or higher backcross progeny plants that comprise the single locus (or trait) and otherwise one or more or all distinguishing characteristics of the spinach according to the invention and/or comprise all of the physiological and morphological characteristics of CALLISTO, when grown in the same environmental conditions.
The backcross progeny in steps (d) and/or (e) may also be backcross progeny which have been selfed one or more times.

The invention provides also for methods of producing EDVs (Essentially Derived Varieties) of CALLISTO which differ from CALLISTO in one, two, three or more morphological and/or physiological characteristics, but which are still genetically closely related to CALLISTO. The relatedness can, for example be determined by fingerprinting techniques (e.g., making use of isozyme markers and/or molecular markers such as SNP markers, AFLP markers, microsatellites, SSR markers, minisatellites, RAPD markers, RFLP markers and others). A plant is “closely related” to CALLISTO if its DNA fingerprint is at least 80%, 90%, 95% or 98% identical to the fingerprint of CALLISTO. In one embodiment AFLP markers are used for DNA fingerprinting (Vos et al. 1995) or other markers, such as microsatellite markers. A closely related plant may have a Jaccard's Similarity index of at least about 0.8, preferably at least about 0.9, 0.95, 0.95, 0.96, 0.97, 0.98 or more (Staub et al 2000). The invention also provides plants and varieties obtained by these methods. In one embodiment, relatedness and whether a variety is an EDV of CALLISTO, may be determined according to the Variety Tracer method of the Nak Tuinbouw (http://www.naktuinbouw.nl/en/topic/identification-using-dna).

In yet another aspect the invention provides a method of producing a spinach plant, comprising selfing a plant designated CALLISTO one or more times, and selecting progeny from said selfing.

Spinach according to the invention, such as the variety CALLISTO, or its progeny or an EDV of CALLISTO, can also be reproduced using vegetative reproduction methods. Therefore, the invention provides for a method of producing plants, or a part thereof, of variety CALLISTO, or of progeny thereof, or of an EDV of CALLISTO, comprising vegetative propagation of variety CALLISTO or of progeny thereof, or of an EDV of CALLISTO. Vegetative propagation comprises regenerating a whole plant from a part of variety CALLISTO (or from its progeny or from an EDV of CALLISTO), such as a cutting, a cell culture or a tissue culture. In vitro cell or tissue culture can also be used to select a somaclonal variant of CALLISTO or of a progeny of CALLISTO.

Accordingly, the invention also provides for a vegetatively propagated plant of variety CALLISTO (or from its progeny or from an EDV of CALLISTO), or a part thereof, having one, two, three or more distinguishing characteristics or all the morphological and/or physiological characteristics of CALLISTO (except for the characteristics differing in the EDV), when grown under the same environmental conditions.

Also included is a cell culture or tissue culture produced from a plant designated CALLISTO. The tissue culture will preferably be capable of regenerating plants capable of expressing all of the physiological and morphological characteristics of CALLISTO or all except one, two or three of the morphological and physiological characteristics of CALLISTO (e.g. a somaclonal variant). The regenerable cells in such tissue cultures may be derived, for example, from embryos, meristems, cotyledons, petioles, pollen, leaves, anthers, roots, root tips, pistil, flower, seed and stalks. Still further, the present invention provides spinach plants regenerated from a tissue culture of the invention, the plants having all the physiological and morphological characteristics of CALLISTO or all except one, two or three of the morphological and physiological characteristics of CALLISTO.

All documents (e.g., patent publications) are herein incorporated by reference in their entirety.

### EXAMPLES

### Development of CALLISTO

The hybrid CALLISTO was developed from a male and female proprietary inbred line of Nunhems. The female and male parents were crossed to produce hybrid (F1) seeds of CALLISTO. The seeds of CALLISTO can be grown to produce hybrid plants and parts thereof (e.g. spinach leaves). The hybrid CALLISTO can be propagated by seeds or vegetative.

The hybrid variety is uniform and genetically stable. This has been established through evaluation of horticultural characteristics. Several hybrid seed production events resulted in no observable deviation in genetic stability. Coupled with the confirmation of genetic stability of the female and male parents the Applicant concluded that CALLISTO is uniform and stable.

### Deposit Information

A total of 2500 seeds of the hybrid variety CALLISTO were deposited according to the Budapest Treaty by Nunhems B.V. on ______, at the American Type Culture Collection (ATCC), 10801 University Boulevard, Manassas, Va. 20110-2209 USA or at the NCIMB Ltd., Ferguson Building, Craibstone Estate, Bucksburn, Aberdeen AB21 9YA, United Kingdom (NCIMB). The deposit has been assigned Accession Number PTA ______ or NCIMB ______. A deposit of CALLISTO and of the male and female parent line is also maintained at Nunhems B.V.

Access to the deposit will be available during the pendency of this application to persons determined by the Director of the U.S. Patent Office to be entitled thereto upon request. Subject to 37 C.F.R. §1.808(b), all restrictions imposed by the depositor on the availability to the public of the deposited material will be irrevocably removed upon the granting of the patent. The deposit will be maintained for a period of 30 years, or 5 years after the most recent request, or for the enforceable life of the patent whichever is longer, and will be replaced if it ever becomes nonviable during that period. Applicant does not waive any rights granted under this patent on this application or under the Plant Variety Protection Act (7 USC 2321 et seq.).

The most similar variety to CALLISTO is PV 0293, a commercial variety from Pop Vriend Seeds. In Table 1 a comparison between CALLISTO and most similar variety PV 0293 is shown based on a trial in the USA. Trial location: Acampo Calif. USA, (coordinates: 38°07′261″N, −121°18′80547″W), USA 2011/2012. Planting date: seeding date Nov. 6, 2011, transplanting date Nov. 4, 2011 and harvested Mar. 13, 2012.

Two replications of 50 plants each, from which at least 15 plants or plant parts were randomly selected to measure characteristics. In Table 1 the USDA descriptors of CALLISTO (this application) and reference PV 0293 (commercial variety) are summarized.

TABLE 1
Comparison between CALLISTO and variety PV 0293 which is
considered the most similar variety.

| USDA CHARACTERISTIC | CALLISTO | PV 0293 |
|---|---|---|
| Species | Spinacia | Spinacia |
| | oleracea L. | oleracea L. |
| PLOIDY | 1 | 1 |
| 1 = Diploid; | | |
| 2 = tetraploid; | | |
| 3 = other | | |
| MATURITY | | |
| Growth Rate | 3 | 1 |
| 1 = slow; | | |
| 2 = medium (Long Standing Bloomsdale); | | |
| 3 = fast (Dixie Market) | | |
| Days from planting to prime market stage | 35 | — |
| PLANT (Prime Market Stage): | | |
| Habit | 2 | 1 |
| 1 = flat (Viroflay); | | |
| 2 = semi-erect (Long Standing Bloomsdale) | | |
| 3 = erect (Virginia Savoy) | | |
| Size | 2 | 2 |
| 1 = small (America); | | |
| 2 = medium; | | |
| 3 = large (Giant Nobel) | | |
| Spread (cm) | 28.6 | 29.3 |
| Height (cm) | 13.1 | 8.2 |
| SEEDLING COTYLEDON | | |
| Width (mm) | 4.6 | 5.5 |
| Length (mm) | 49.2 | 43.9 |
| Tip | 1 | 2 |
| 1 = pointed; | | |
| 2 = rounded | | |
| Color | 2 | 2 |
| 1 = light green; | | |
| 2 = medium green; | | |
| 3 = dark green; | | |
| 4 = other | | |
| Color Chart | RHS | RHS |
| Color Chart Value | Green | Yellow- |
| | 138A | Green |
| | | 144A |
| LEAF (First Foliage Leaves) | | |
| Shape | 3 | 2 |
| 1 = elliptic | | |
| 2 = circular; | | |
| 3 = ovate; | | |
| 4 = other | | |
| Base | 1 | 1 |
| 1 = V-base; | | |
| 2 = straight; | | |
| 3 = lobed | | |
| Tip | 2 | 1 |
| 1 = round; | | |
| 2 = round-pointed; | | |
| 3 = pointed | | |
| Margin | 2 | 3 |
| 1 = flat; | | |
| 2 = slightly curled; | | |
| 3 = curled under | | |
| Upper Surface Color | 2 | 2 |
| 1 = light green (Hollandia); | | |
| 2 = medium green (Giant Nobel); | | |
| 3 = dark green (Long Standing Bloomsdale) | | |
| Color Chart | RHS | RHS |
| Color Chart Value | Green | Green |
| | 141A | 141A |
| Lower Surface Color | 2 | 2 |
| 1 = lighter; | | |
| 2 = same; | | |
| 3 = darker | | |
| Color Chart | RHS | RHS |
| Color Chart Value | Green | Green |
| | 143A | 143A |
| LEAF (Prime Market Stage) | | |
| Surface | 1 | 2 |
| 1 = smooth (Viroflay); | | |
| 2 = semi-savoy (Northland); | | |
| 3 = savoy (Virginia Savoy) | | |
| Shape | 5 | 2 |
| 1 = elliptic | | |
| 2 = circular; | | |
| 3 = ovate; | | |
| 4 = three sided; | | |
| 5 = five-sided; | | |
| 6 = arrow-shaped; | | |
| 7 = asymmetrical | | |
| Base | 3 | 3 |
| 1 = V-base; | | |
| 2 = straight; | | |
| 3 = lobed | | |
| Tip | 2-3 | 1 |
| 1 = round; | | |
| 2 = round-pointed; | | |
| 3 = pointed | | |
| Margin | 2 | 3 |
| 1 = flat; | | |
| 2 = slightly curled; | | |
| 3 = curled under; | | |
| 4 = curled up | | |
| Upper Surface Color | 2 | 2 |
| 1 = light green (Hollandia); | | |
| 2 = medium green (Giant Nobel); | | |
| 3 = dark green (Long Standing Bloomsdale) | | |
| 4 = dull green (Northland) | | |
| Color Chart | RHS | RHS |
| Color Chart Value | Yellow | Yellow |
| | Green | Green |
| | 147A | 147B |
| Lower Surface Color (compared to upper | 1 | 1 |
| surface color) | | |
| 1 = lighter; | | |
| 2 = same; | | |
| 3 = darker | | |
| Color Chart | RHS | RHS |
| Color Chart Value | Yellow | Yellow |
| | Green | Green |
| | 146A | 146B |
| Luster | 1 | 2 |
| 1 = glossy | | |
| 2 = dull | | |
| Blade Size | 3 | 3 |
| 1 = small (Long Standing Bloomsdale); | | |
| 2 = medium (Virginia Savoy); | | |
| 3 = large (Giant Nobel) | | |
| Blade Lobing | 2 | 2 |
| 1 = not lobed; | | |
| 2 = lobed | | |
| Petiole Color | 3 | 3 |
| 1 = white; | | |
| 2 = light yellow; | | |
| 3 = light green; | | |
| 4 = medium green | | |
| Color Chart | RHS | RHS |
| Color Chart Value | Yellow | Yellow |
| | Green | Green |
| | 146A | 146C |
| Petiole Red Pigmentation | 2 | 2 |
| 1 = present; | | |
| 2 = absent | | |
| Petiole Length to the Blade (cm) | 4.4 | 3.3 |
| Petiol Length | 2 | 1 |
| 1 = short; | | |
| 2 = medium; | | |
| 3 = long (Viroflay) | | |
| Petiole Diameter (mm) | 5.9 | 6.4 |
| Petiole Diameter | 2 | 3 |
| 1 = small; | | |
| 2 = medium; | | |
| 3 = large (Giant Nobel) | | |
| SEED STALK DEVELOPMENT | | |
| Start of Bolting (10% of plants): | 2-3 | 3 |
| 1 = Early (Dixie Market); | | |
| 2 = Medium (Long Standing Bloomsdale); | | |
| 3 = Late (Norgreen) | | |
| Height of Stalk (cm) | — | — |
| Leaves on Stalk of Female Plant: | — | — |
| 1 = Few or None | | |
| 2 = Many | | |
| Leaves on Stalk of Male Plant: | — | — |
| 1 = Few or None | | |
| 2 = Many | | |
| Plants that are Female: | — | — |
| 1 = 0-10% | | |
| 2 = 11-35% | | |
| 3 = 36-65% | | |
| 4 = 66-90% | | |
| 5 = 91-100% | | |
| Plants that are Male: | — | — |
| 1 = 0-10% | | |
| 2 = 11-35% | | |
| 3 = 36-65% | | |
| 4 = 66-90% | | |
| 5 = 91-100% | | |
| Plants that are Monoecious: 1 = 0-10% | — | — |
| 2 = 11-35% 3 = 36-65% 4 = 66-90% | | |
| 5 = 91-100% | | |
| SEED: | | |
| Surface: | 1 | 1 |
| 1 = Smooth; | | |
| 2 = Prickly | | |
| DISEASE REACTION: (0 = Not Tested, 1 = | 0 | 0 |
| Susceptible, 2 = Resistant) | | |
| Downy Mildew (Peronospora spinaciae) | 2 | 2 |
| Race 1-13 | | |
| Downy Mildew (Peronospora spinaciae) | 2 | 1 |
| Race 14 | | |
| Fusarium Wilt (Fusarium oxysporum f. sp. | 0 | 0 |
| spinaciae) | | |
| White Rust (Albugo Occidentalis) | 0 | 0 |
| Curly Top Virus | 0 | 0 |
| Cucumber Mosaic Virus | 0 | 0 |
| WINTER HARDINESS: | | |
| Hardiness: | 2 (Coastal | — |
| 1 = Not Hardy | CA) | |
| 2 = Moderate | | |
| 3 = Hardy | | |

These are typical values. Values may vary due to environment. Other values that are substantially equivalent are also within the scope of the invention.
“—” means not determined.

### REFERENCES

The following references, to the extent that they provide exemplary procedural or other details supplementary to those set forth herein, are specifically incorporated herein by reference:
http://www.ams.usda.gov/AMSv1.0/getfile?dDocName=STELDEV3003749.
http://www.upov.int/edocs/tgdocs/en/tg055.pdf.
http://www.rhs.org.uk/Plants/RHS-Publications/RHS-colour-charts.
U.S. Pat. No. 5,268,526.
U.S. Pat. No. 5,792,921.
U.S. Pat. No. 6,194,638.
U.S. Pat. No. 7,041,875.
U.S. Pat. No. 7,081,363.
U.S. Pat. No. 7,098,378.
Staub et al 2000, Euphytica 115: 225-241.
Vos et al. 1995, Nucleic Acid Research 23: 4407-4414.
http://www.naktuinbouw.nl/en/topic/identification-using-dna.

1. A spinach plant, designated CALLISTO, or part thereof a representative sample of seeds of which having been deposited under Accession Number NCIMB ______.

2. A seed of CALLISTO, a sample of seed of said variety having been deposited under ATCC Accession Number ______.

3. A plant grown from the seed of claim 2.

4. A plant part of the plant of claim 3.

5. The plant part of claim 4, wherein said part is selected from the group consisting of a seed, pollen, an ovule, a leaf, a petiole, a flower, and a cell.

6. A variant spinach plant derived from the plant of claim 1, which variant plant has all the morphological and/or physiological characteristics of the plant of claim 1, except that it significantly differs from the plant of claim 1 in one physiological and/or morphological characteristic.

7. A spinach plant, or a part thereof, which does not significantly differ from the lettuce plant of claim 1 in any of the distinguishing characteristics consisting of: 1) shape of leaf at prime market stage; 2) tip of leaf at prime market stage; 3) color and luster of leaf at prime market stage; 4) average petiole length and width of the leaf at prime market stage; 5) margin of the leaf at prime market stage; 6) average plant height at prime market stage; 7) growth rate of the plant and 8) average cotyledon width and length.

8. A cell culture or tissue culture of the spinach plant of claim 1 CALLISTO, a sample of seed of said hybrid variety having been deposited under NCIMB Accession Number ______.

9. The cell or tissue culture according to claim 8, comprising cells or protoplasts from a plant part selected from the group consisting of embryos, meristems, cotyledons, pollen, leaves, anthers, roots, root tips, shoots, pistil, flower, seed and stalks.

10. A spinach plant regenerated from the cell or tissue culture of claim 9, wherein the regenerated plant expresses all of the physiological and morphological characteristics of spinach hybrid variety CALLISTO, a sample of seed of said hybrid variety having been deposited under NCIMB Accession Number ______.

11. A spinach plant regenerated from the cell or tissue culture of claim 9, wherein the regenerated plant expresses all, except one, of the physiological and/or morphological characteristics of spinach hybrid variety CALLISTO, a sample of seed of said hybrid variety having been deposited under NCIMB Accession Number ______.

12. A method of producing seed, comprising crossing the plant of claim 3 with itself or a second spinach plant and allowing seed to form.

13. The method of claim 12, comprising crossing the plant of spinach hybrid variety CALLISTO with a spinach plant of a different genotype relative to CALLISTO.

14. An F1 hybrid seed produced by the method of claim 13.

15. An F1 hybrid plant produced by growing the seed of claim 14.

16. A method for producing a seed of a CALLISTO-derived spinach plant comprising the steps of:
(a) crossing a spinach plant of hybrid variety CALLISTO, a sample of seed of said variety having been deposited under NCIMB Accession Number ______, with a second spinach plant; and
(b) allowing seed of a CALLISTO-derived spinach plant to form.

17. The method of claim 16, further comprising the steps of:
(c) crossing a plant grown from said CALLISTO-derived spinach seed with itself or a second spinach plant to yield additional CALLISTO-derived spinach seed; and
(d) growing said additional CALLISTO-derived spinach seed of step (c) to yield additional CALLISTO-derived spinach plants; and
(e) repeating the crossing and growing steps of (c) and (d) to generate further CALLISTO-derived spinach plants.

18. A method of vegetatively propagating a plant of spinach variety CALLISTO comprising the steps of:
(a) collecting cells or tissue capable of being propagated from a plant of spinach variety CALLISTO, a sample of seed of said variety having been deposited under NCIMB Accession Number ______;
(b) cultivating said cells or tissue to obtain proliferated shoots; and
(c) rooting said proliferated shoots to obtain rooted plantlets.

19. The method of claim 18, further comprising growing plants from said rooted plantlets.

20. A method of introducing a desired trait into spinach variety CALLISTO comprising:
(a) crossing a plant of variety CALLISTO, a sample of seed of said variety having been deposited under NCIMB Accession Number ______, with a second spinach plant that comprises a desired trait to produce F1 progeny and optionally selfing said F1 progeny one or more times to produce F2 or further selfing progeny;
(b) selecting an F1, F2 or further selfing progeny that comprises the desired trait;
(c) crossing the selected F1, F2 or further selfing progeny with a plant of variety CALLISTO to produce backcross progeny;
(d) selecting backcross progeny comprising the desired trait and the physiological and morphological characteristic of spinach variety CALLISTO; and
(e) repeating steps (c) and (d) three or more times in succession to produce selected higher backcross progeny that comprise the desired trait.

21. A spinach plant produced by the method of claim 20, wherein the spinach plant comprises essentially all of the physiological and morphological characteristics of spinach variety CALLISTO, a sample of seed of said variety having been deposited under NCIMB Accession Number ______, and the desired trait.

22. A progeny plant of the plant of claim 3 that comprises all of the physiological and morphological characteristics of spinach variety CALLISTO, a sample of seed of said variety having been deposited under NCIMB Accession Number ______.

23. A seed that produces the plant of claim 22.

24. An Essentially Derived Variety of CALLISTO having one, two or three physiological and/or morphological characteristics which are different from those of CALLISTO and which otherwise has all the physiological and morphological characteristics of CALLISTO, wherein a representative sample of seed of variety CALLISTO has been deposited under Accession Number NCIMB ______.

25. A method of determining the genotype of the plant of claim 3, comprising obtaining a sample of nucleic acids from said plant and detecting in said nucleic acids a plurality of polymorphisms.

26. The method of claim 25, further comprising the step of storing the results of the step of detecting the plurality of polymorphisms on a computer readable medium.

27. A method of producing spinach comprising:
(a) obtaining the plant of claim 3, and
(b) collecting leaf tissue from the plant.
